(19) United States
(12) Patent Application Publication
(10) Pub. No.: US 2024/0115541 A1
(43) Pub. Date: Apr. 11, 2024

(54) METHOD FOR TREATING CEREBRAL APOPLEXY AND HYPERBILIRUBINEMIA BY COMPOUND FOR INHIBITING COMBINATION OF BILIRUBIN AND TRPM2 CHANNEL

(71) Applicant: SHANGHAI SIXTH PEOPLE'S HOSPITAL, Shanghai (CN)

(72) Inventors: Shankai YIN, Shanghai (CN); Haibo SHI, Shanghai (CN); Hanwei LIU, Shanghai (CN); Ke LAI, Shanghai (CN); Luyang WANG, Shanghai (CN)

(21) Appl. No.: 18/191,440

(22) Filed: Mar. 28, 2023

(30) Foreign Application Priority Data
Oct. 10, 2022 (CN) 2022211237436.3

(51) Int. Cl. A61K 31/277; A61P 7/00

(52) U.S. Cl. CPC A61K 31/277; A61P 7/00

(57) ABSTRACT

The invention provides a method for treating cerebral apoplexy and hyperbilirubinemia by the compound or reagent for competitively inhibiting or blocking combination of bilirubin and the TRPM2 channel. The compound or the reagent is competitively combined with the D1069 residue of TRPM2 channel (the analogous D1066 residue of the mouse TRPM2). It is verified that bilirubin can serve as an extracellular endogenous agonist to be directly combined with the TRPM2 channel to aggravate brain tissue damage caused by stroke and hyperbilirubinemia, K928 and/or D1069 residues are key amino acid residues for playing roles. When the K928 and/or D1069 residues are mutated or competitively combined, the bilirubin is prevented from activating the TRPM2 channel; wherein D1069 residue mutation can effectively antagonize the nerve injury effect caused by bilirubin, which would be a therapeutic target for relieving and treating ischemic stroke and hyperbilirubinemia related brain damage.

### BACKGROUND OF THE INVENTION

### 1. Field of the Invention

The invention relates to the technical field of structural biology and biomedicine, and more particularly, to the discovery and application of bilirubin-TRPM2 binding sites and a method for treating cerebral apoplexy and hyperbilirubinemia by the compound for antagonizing or inhibiting the combination of bilirubin and the TRPM2 channel.

### 2. Description of the Related Art

Cerebral apoplexy (also called stroke) is one of the leading causes of death and disability in adults. The most typical pathological change of cerebral apoplexy is the interruption of cerebral blood and oxygen supply, leading to the necrosis of neurons and brain injury. During cerebral apoplexy, cerebral ischemia leads to a massive release of glutamate which activates N-methyl-D-aspartate receptors (NMDARs) and induces Ca²⁺ influx through these ionotropic channels to overload neurons and destroy their Ca²⁺ homeostasis. This triggers a plethora of cascades that work synergistically to induce secondary neuronal death induced by acidosis, reactive oxygen species (ROS) and nitrogen species (RNS), which activate other nonselective cation channels such as acid sensing ion channels (ASICs) and transient receptor potential melastatin (TRPM) channels. Despite extensive work on ischemic damage, few drugs offering significant benefits in preclinical studies have successfully advanced to the stage of approved therapeutics for stroke patients, rationalizing the pressing need to identify the mechanisms of stroke and potential new targets for effective treatments of ischemic stroke.

Previous studies suggested that cerebral apoplexy appears to be associated with an elevation of bilirubin. As the end-product of heme catabolism in mammals, bilirubin is typically low (<1 mg/dL) but when the concentration exceeds certain levels (i.e., 3 mg/dL), it can be visually observed as hyperbilirubinemia or jaundice. Jaundice is known to cause damage to sensory, motor and cognitive functions, especially in the early developing brain. Excessive bilirubin induces oxidative stress of endoplasmic reticulum (ER), triggering inflammatory responses, hyperexcitation, Ca²⁺ overload and neuronal injury by indirectly modulating many ion channels and intracellular Ca²⁺ release. More critically, previous studies have shown that increased levels of bilirubin have been found in patients with acute ischemic stroke and is associated with the severity of stroke. Although a lot of research work has been carried out on the neurotoxicity of bilirubin, neither specific targets nor the mechanisms of bilirubin-induced neurotoxicity are known. Extensive work from the inventor's previous studies and others in a variety of neurons all converge to the idea that bilirubin-induced hyperexcitation and Ca²⁺ overload are the leading causes of neuronal injury. However, it is unknown whether bilirubin can be a direct agonist for non-elective cation channels known to initiate Ca²⁺-dependent signaling cascade that precedes ultimate neuronal death in stroke.

TRPM2 channels are abundantly expressed in the brain and serve as the oxidative stress sensor in vivo and can be activated by ADP-ribose (ADPR) and/or the oxidative stress generated by ADPR. More importantly, TRPM2 channel is one of several highly Ca²⁺ permeable non-selective cation channels and mediates a variety of physiological and pathological processes by regulating multiple intracellular signaling pathways. Canonically, NMDAR-dependent Ca²⁺ influx and ADPR rise lead to subsequent activation of multiple TRP channels including TRPM2. These pathways have been viewed as the primary routes for ischemia and reperfusion-induced cell death in stroke. The purpose of this invention is to clarify the relationship between bilirubin and stroke-related ion channels (exactly TRPM2), reveal the key interaction sites which could be targeted inhibition, so as to provide a treatment scheme for stroke and hyperbilirubinemia.

### SUMMARY OF THE INVENTION

In order to overcome the defects in the prior art, the invention provides a method for treating cerebral apoplexy and hyperbilirubinemia by the compound for inhibiting the combination of bilirubin and the TRPM2 channel. Specifically, the endogenous metabolite bilirubin directly binds to K928 and/or D1069 residue(s) of TRPM2 channel as an extracellular agonist, aggravating ischemic brain injury caused by stroke. Compound structurally similar with A23 can competitively bind to this site and antagonize the neurotoxicity of bilirubin.

For the above-mentioned purposes, the present invention provides the following technical solution:

In the first aspect of the invention, based on the discovery of bilirubin-TRPM2 binding sites, there is provided a method for treating cerebral apoplexy and hyperbilirubinemia, the method comprising administering to a subject in need thereof a therapeutically effective amount of a medicine comprising the compound or the reagent for competitively inhibiting or blocking combination of bilirubin and the TRPM2 channel.

Furthermore, the compound or the reagent is competitively combined with the K928 and/or D1069 residue(s) of the TRPM2 channel to provide therapeutic effect.

Furthermore, K928 and/or D1069 mutation deprives the activating effect of bilirubin and its derivatives.

Furthermore, the mutation is a substitution mutation.

Furthermore, the TRPM2 channel has a substitution mutation of the K928 and/or D1069 residue(s).

Furthermore, the TRPM2 channel protein has a substitution mutation of the K928 and D1069 residues.

In a second aspect of the invention, there is provided a medicine for treating cerebral apoplexy and hyperbilirubinemia, the medicine comprising a compound or a reagent for competitively inhibiting or blocking combination of bilirubin and the TRPM2 channel, the compound or the reagent acting as a main active component.

Furthermore, the compound or the reagent prevents bilirubin from activating the TRPM2 channel, the compound or the reagent is competitively combined with the K928 and/or D1069 residue(s) of the TRPM2 protein and thus prevents the bilirubin from activating the TRPM2 channel.

Furthermore, the medicine further comprises a pharmaceutically acceptable carrier or a pharmaceutically acceptable excipient.

Furthermore, routes for administration of the medicine comprise oral, transdermal, intramuscular, subcutaneous or intravenous injections.

Compared with the prior art, the present invention has the following beneficial effects that it is verified that bilirubin can serve as an extracellular endogenous agonist to be directly combined with the TRPM2 channel to aggravate brain damage caused by cerebral apoplexy, K928 and D1069 are key amino acid residues for bilirubin-TRPM2 binding, and when the K928 and/or D1069 residue(s) are mutated or competitively combined, the bilirubin is prevented from activating the TRPM2 channel; and more importantly, D1069 residue substitution mutation (the analogous D1066 residue of the mouse TRPM2) can effectively antagonize the brain injury caused by bilirubin, and a therapeutic target is provided for relieving and treating ischemic stroke and hyperbilirubinemia.

### BRIEF DESCRIPTION OF THE DRAWINGS

FIG. 1A to FIG. 1F illustrate that hyperbilirubinemia exacerbates brain damage in adult ischemia models in vivo; wherein FIG. 1A to FIG. 1B show representative images of brain sections by 2,3,5-triphenyltetrazolium chloride (TTC) staining of tMCAO Trpm^{2+/+} and Trpm^{2-/-} adult mice with intraperitoneal injection of saline (Ctrl) and bilirubin (Bil) and summary data showing normalized infarct volumes of Ctrl and Bil group in Trpm^{2+/+} and Trpm^{2-/-} mice; FIG. 1C to FIG. 1D illustrate Spearman correlation analyses of serum/cerebrospinal fluid (CSF) total bilirubin (TB) concentration and the infarct volume in tMCAO mice. In Trpm^{2+/+} mice, both serum and CSF TB were significantly correlated with the infarct volume, but these correlations were absent in Trpm^{2-/-} mice; FIG. 1E to FIG. 1F show serum and CSF unconjugated bilirubin (UCB) concentration of Ctrl and Bil group in Trpm^{2+/+} and Trpm^{2-/-} animals after tMCAO surgery;

FIG. 2A to FIG. 2G show that bilirubin can directly bind to and activate the TRPM2 channel; wherein, FIG. 2A to FIG. 2B show the representative time course of the currents activated by voltage ramps from −100 to +100 mV and the amplitudes measured at +80 mV (strawberry circle) and −80 mV (black circle) are plotted against time after membrane breakthrough. Example ramp current traces before and after bilirubin application and co-application with FFA were transformed into I-V relationships and overlayed on the bottom panels; FIG. 2C to FIG. 2E show the representative single-channel recording showing bilirubin (300 nM) directly activated TRPM2 currents (holding potential −40 mV), and all-points amplitude histogram of single channel current amplitude (0.2 pA/bin) before and after perfusing bilirubin was shown in the middle panel. Statistical results of opening probability when perfusion of bilirubin and FFA was shown in the bottom; FIG. 2F to FIG. 2G show the mean single-channel current amplitudes activated by bilirubin from −80 mV to +80 mV are plotted against each voltage and fit with linear regression to yield single-channel conductance of 76.29±7.85 pS, which is consistent with the conductance of the normal TRPM2 channel;

FIG. 3A to FIG. 3I show that bilirubin activates TRPM2 channel by binding to a cavity in transmembrane region near the Ca²⁺ binding site; wherein, FIG. 3A to FIG. 3B show an overview of the molecular docking for bilirubin-bound hTRPM2. A close-up view of the bilirubin binding site is shown in the inset. Individual TRPM2 subunits are colored cyan and grey whereas bilirubin is highlighted with yellow, respectively. Chemical structure and the EM density of bilirubin are shown; FIG. 3C to FIG. 3D show bilirubin binds a deep cavity of TRPM2 between S3, S5 and TRP helix, Ca²⁺ is colored green (Left panel). 2D diagram showing bilirubin interacts with TRPM2 through a mixture of hydrogen bonds (H-bonds), salt bridges and n-n interaction (Right panel); FIG. 3E to FIG. 3F show representative time-course of currents at +80 mV and −80 mV activated by 500 μM ADPR in K928A/D1069A double mutant. I-V plots are shown in the right panels; FIG. 3G shows the amplitude of currents activated by ADPR in WT TRPM2 channel and K928A/D1069A double mutant; FIG. 3H to FIG. 3I show representative time-course of currents at +80 mV and −80 mV activated by 9 μM bilirubin in K928A/D1069A double mutant. I-V plots are shown in the right panel;

FIG. 4A to FIG. 4L show A23 antagonized the damage of bilirubin by specifically blocking the TRPM2 channel; FIG. 4A to FIG. 4D molecular docking showing the specific recognition of bilirubin disodium salt (BDS) (A) and A23 (B)-bound hTRPM2. 3D diagrams showing that BDS and A23 docking to the cavity where bilirubin binding the TRPM2 channel. 2D diagrams showing BDS and A23 interacts with TRPM2 through H-bonds and salt bridges. (docking score in a closed state: A23-8.57>bilirubin-5.04>BDS-4.47; docking score in an open state: bilirubin-7.84>A23-7.38>BDS-5.60); FIG. 4E to FIG. 4H show that TRPM2 currents activated by BDS which was completely antagonized by A23, and when HEK293 cells incubated with A23 for 20 min in advance, BDS failed to activate the TRPM2 channel; FIG. 4I to FIG. 4J show the dose-response curves for A23 to block TRPM2 currents evoked by bilirubin and BDS, respectively. The concentration for A23 to produce 50% blockade (IC 50) were estimated to be 0.92 and 0.72 μM; FIG. 4K to FIG. 4L show the representative immunofluorescence staining results and mortality ratio of bilirubin induced death of cortical neurons in adult Trpm2^{+/+} brain slices. Prominent calcein-am staining (green) of nuclei marked the population of live neurons and PI staining (purple) of nuclei marked the population of dead neurons;

FIG. 5A to FIG. 5D show that D1066A knock-in (KI) mice phenocopy TRPM2 knockout mice in attenuating bilirubin-dependent effects on neurotoxicity; wherein, FIG. 5A to FIG. 5B show representative images of brain sections by TTC staining and normalized infarct volumes of D1066A KI mice adult with intraperitoneal injection of saline (Ctrl) and bilirubin (Bil); FIG. 5C to FIG. 5D comparable serum and CSF UCB concentration of Ctrl and Bil group in D1066A KI mice 24 hours after tMCAO surgery, respectively.

### DETAILED DESCRIPTION

The invention provides the key amino acid residues in bilirubin-TRPM2 interaction and a potential method for treating cerebral apoplexy and hyperbilirubinemia by the compound for inhibiting the combination of bilirubin and the TRPM2 channel. The invention will be described in details with reference to particular embodiments and accompanying drawings, so as to better understand the invention. However, the invention is not limited to the particular embodiments illustrated hereinafter.

Methods in the examples are conventional methods, unless otherwise specified. Reagents used herein are conventional commercially-available reagents or reagents prepared according to the conventional methods, unless otherwise specified.

### Example 1

This example explores the effect of hyperbilirubinemia on brain damage caused by ischemic stroke in an animal model in vivo. In the following, specific experimental steps and results are illustrated:

1. Construction of transient middle cerebral artery occlusion (tMCAO) animal model

a) Wild-type (Trpm2^{+/+}) and TRPM2 knockout (Trpm2^{-/-}) mice, aged between 6 and 8 weeks and weighing 20-25 g, both male and female were used in this experiment. Anesthesia was induced by using a 2% isoflurane-oxygen mixture for induction and 1.5% for maintenance.

b) Bilirubin was first dissolved in 1 M NaOH solution, and its pH value was adjusted back to 7.4-8.0 by titrations with 1 M HCl. Before the operation, bilirubin (50 μg/g) or saline were injected intraperitoneally.

c) tMCAO model was achieved by inserting a monofilament suture into the right MCA via the internal carotid artery. MCA embolization lasted for 30 min, and body temperature was maintained at 37° C. using a heated blanket. Adequate ischemia was confirmed by continuous laser Doppler flowmetry (moor FLPI-2). Animals that did not have a significant reduction of blood flow less than 30% baseline values during MCAO were excluded. When the surgery was finished, mice were placed on another 37° C. heating blanket till they regained consciousness and then returned to the cage. The blood pressure, body temperature and serum biochemical markers were monitored in mice before and after tMCAO surgery.

2. Detection of UCB concentration in serum and cerebrospinal fluid (CSF) of animal model

a) Trpm2^{+/+}, Trpm2^{-/-} and D1066A C57BL/6 mice were anesthetized by intraperitoneal injection of pentobarbital (55 mg/kg), and then fixed in the stereotactic setup. The skin above the skull from the base of the neck up to in between the eyes was cut open. A hole directly above the right ventricle was made with a grinding drill to allow insertion of the micro syringe into the lateral ventricle (coordinates: 1.1 mm laterally to the right and 0.5 mm posterior of the bregma, 2.5 mm deep). CSF was collected by using the micro injection pump (speed: 0.2 μl/min), after which the syringe was withdrawn, and the skin was sutured. The mice were removed from the stereotactic setup and 50 μl blood was taken through the orbital venous plexus. Mice were then placed on 37° C. heating blanket till they regained consciousness and returned to the cage.

b) Detection of bilirubin concentration in serum and cerebrospinal fluid: 24 hours after tMCAO, animals were anesthetized using a 2% isoflurane-oxygen mixture. CSF and blood were collected in the same way before sacrifice. All samples were centrifuged at 3000 r/min. Total bilirubin concentration of supernatants was measured with the Bilirubin Reagent Kit (Sigma). Samples were transferred to a 96-plates and mixed with reaction solution before measurements of their absorbance were made at 530 nm 10 minutes later. UCB concentration was measured with UnaG, a bilirubin-inducible fluorescent protein from Japanese eel muscle. For standard calibration curve of fluorescence intensity, a 100 μl reaction mixture containing 50 μl UnaG solution (1 μM) and 50 μl artificial bilirubin solution with concentration gradient (1.4278 μM, 0.7139 μM, 0.3573 μM, 0.1785 μM, 0.0893 μM and 0 μM) was prepared. After 10 minutes of reaction, the fluorescence intensity was detected by microplate reader with fluorescence filters for excitation and emission wavelengths of 485 and 528 nm, respectively. Serum and CSF samples were diluted 20-fold with PBS and fluorescence intensity was measured. The UCB levels were extrapolated from the standard curve.

3. Detection of cerebral infarct volume in animal models of ischemic stroke

24 hours after tMCAO, animals were anesthetized using a 2% isoflurane-oxygen mixture. Brains were extracted and coronally sectioned into 1 mm slices, which were then stained with 2% TTC for 20 min at 37° C. The infarct volume was analyzed using ImageJ and the infarct volumes were calculated according to the following formula: Corrected infarct volume (%)=[contralateral hemisphere volume−(ipsilateral hemisphere volume−infarct volume)]/contralateral hemisphere volume×100%.

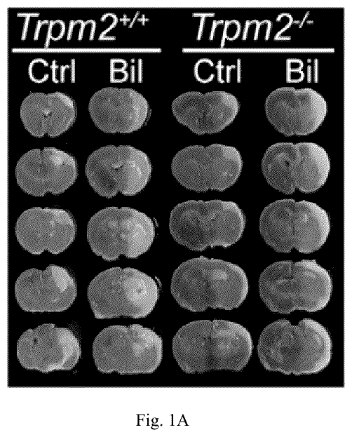

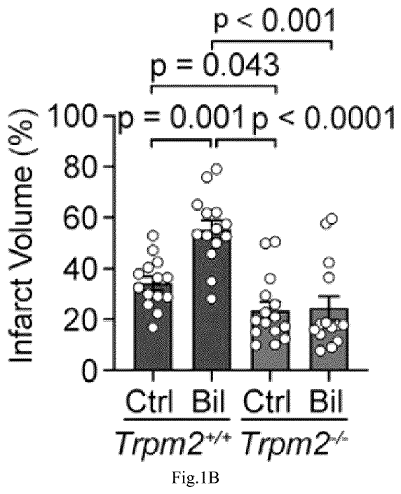

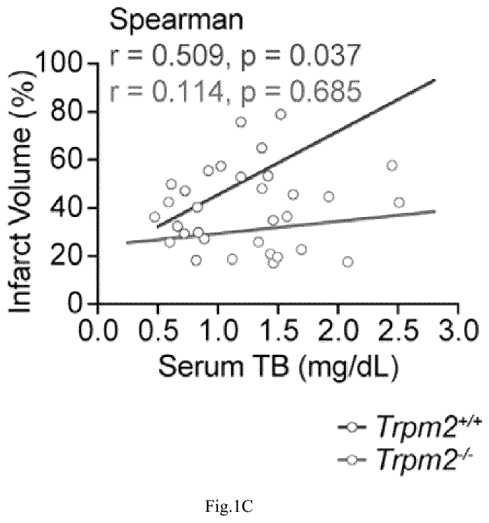

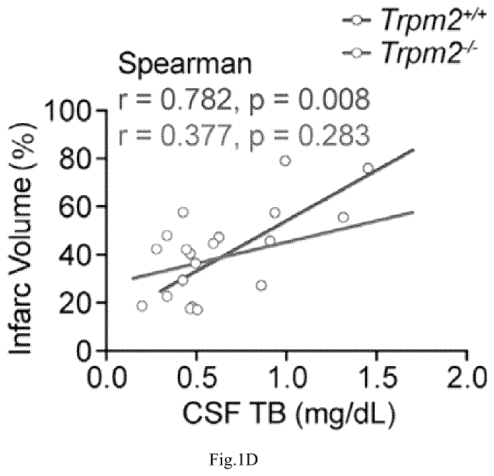

As shown in FIG. 1A to FIG. 1B, In Trpm2^{+/+} mice with tMCAO, bilirubin increased the infarct volume. In contrast, the infarct volume in Trpm2^{-/+} mice did not change by bilirubin injection; and both serum and CSF TB showed significant correlations with the infarct volume in Trpm2^{+/+} animals, despite similar increases of TB levels in blood, neither the serum nor CSF bilirubin levels correlated with infarct volume in Trpm2^{-/-} mice (FIG. 1C to FIG. 1D). Moreover, in Trpm2^{+/+} mice, the UCB concentration of Bil group was also much higher than that of Ctrl group (both in serum and CSF) 24 hours after tMCAO, while the level of UCB in Trpm2^{-/-} mice did not change (FIG. 1E to FIG. 1F).

In conclusion, ischemia-reperfusion during stroke aggravates brain injury and increases the concentration of bilirubin in some way that involve TRPM2.

### Example 2

In this embodiment, the patch-clamp electrophysiology is used to study whether bilirubin can directly activate the TRPM2 channel Specific experimental steps and results are as follows.

1. Extracellular solution (in mM): 145 NaCl, 5.6 KCl, 2 MgCl₂, 1.2 CaCl₂, 10 HEPES, 10 glucose and pH adjusted to 7.4 by NaOH (290-320 mOsm).

2. Pipette solution contains (in mM): 147 NaCl, 1 MgCl₂, 1 EGTA and 10 HEPES with pH 7.4 and osmolality 290-320 mOsm.

3. Cell culture and transfection: Human embryonic kidney 293 (HEK293) cells with tetracycline-inducible expression of human TRPM2 channel (hTRPM2) were cultured in a mixed medium containing DMEM/F-12 and 10% fetal bovine serum as well as blasticidin (50 μg/ml) and zeocin (0.4 mg/ml). The expression of hTRPM2 was induced by substituting blasticin and zeocin for tetracycline (1 μg/ml) 24-48 hours before use. TRPM2 channel mutants were transiently expressed in HEK293T cells by transfection with using Lipofectamine 3000. Briefly, HEK293T cells were transiently transfected with cDNAs encoding the mutant hTRPM2 channel. The cDNA for GFP was co-transfected as a marker for identification of the transfected cells for electrophysiological experiments 16-24 hours after transfection. All the cells were seeded on 96-well coverslips (3×3 mm) and cultured at 37° C. under a humidified atmosphere containing 5% CO2.

4. Whole-cell and ingle-channel patch-clamp recording: the coverslips were placed in a culture dish (35×35 mm, Sorfa) containing HEPES based extracellular solution (ECS) and recorded by patch-clamp technique. Cells were visualized by using the phase-contrast mode of an inverted microscope and the patch pipette was positioned on the cells using a motorized micromanipulator under the microscope. The transfected cells were voltage-clamped at 0 mV and subjected to voltage ramps from −100 mV to +100 mV (500 ms duration) every 5 s. The basal ramp current (i.e., the trace when the leak current is stable after membrane rupture) before channel activation were used for leak subtraction of all subsequent ramp currents. Each coverslip was recorded only once with different drug exposures. Single-channel recordings were carried out under outside-out configuration, using pipettes with resistance of 6-8 MΩ. The excised patches were voltage-clamped from −80 mV to +80 mV (in 20 mV increasement) to generate the current-voltage (I-V) relationship of single channel currents from which their main conductance was calculated from the slope by linear regression of the mean current amplitude at different potentials.

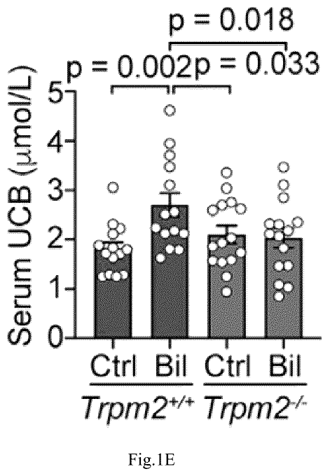

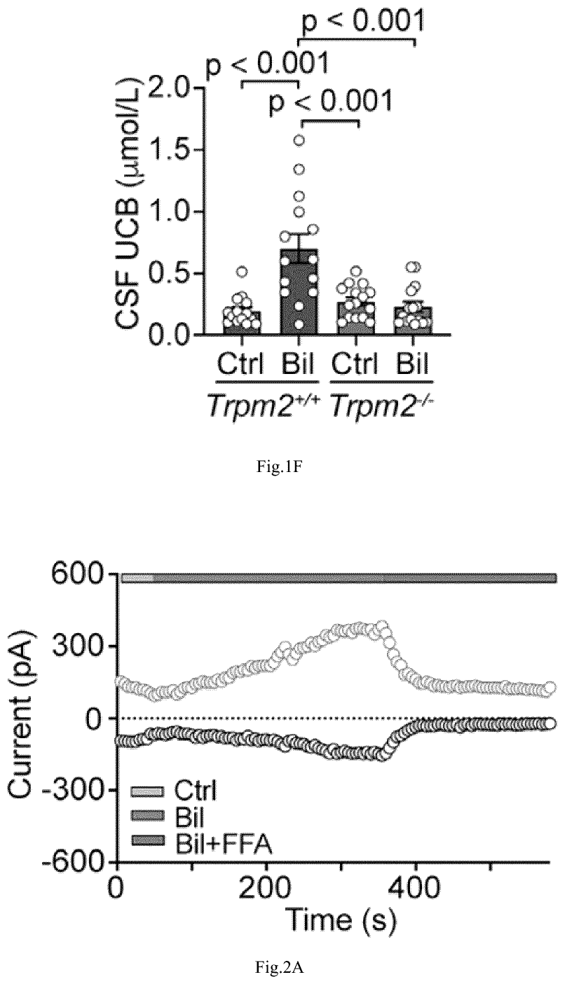

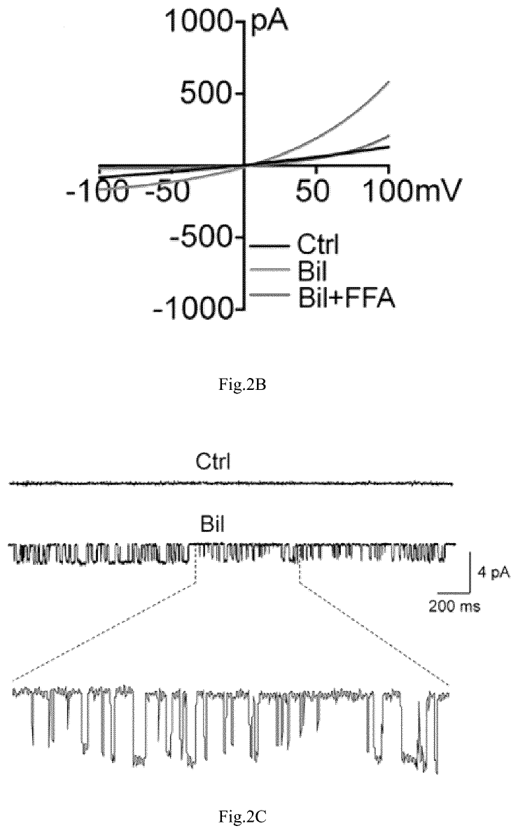

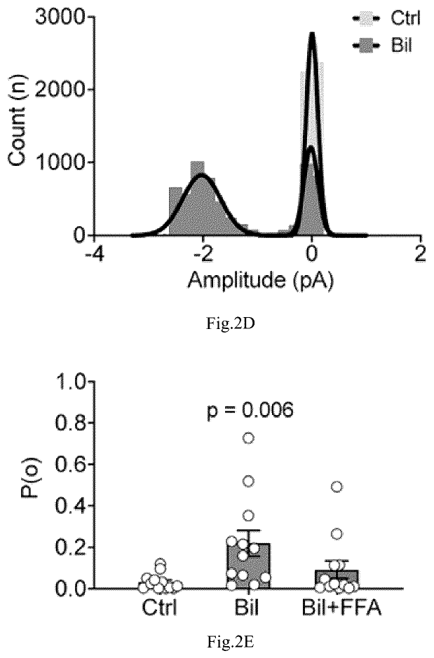

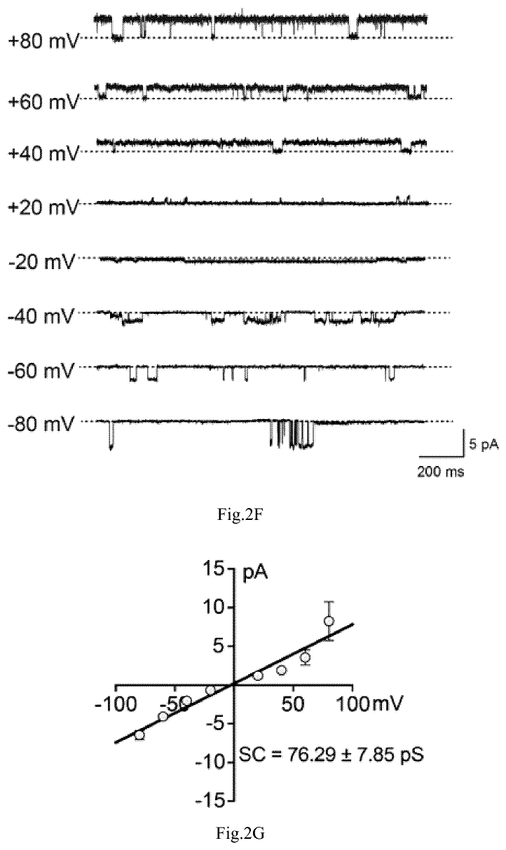

As shown in FIG. 2A to FIG. 2B, in the whole-cell patch-clamp recordings, bilirubin (9 μM) increased the magnitude of the TRPM2 currents evoked by voltage ramps (−100 to +100 mV), as shown by time-dependent increases in current amplitude at both negative and positive potentials without affecting the reversal potential (˜0 mV) of the current-voltage (I-V) curves. Also, bilirubin (300 nM) elicited single channel openings in membrane patches from induced HEK-hTRPM2 cells: these channels exhibited biophysical properties and pharmacological sensitivity to the blocker flufenamic acid (FFA) characteristic of the TRPM2 channel (FIG. 2C to FIG. 2G). As shown in FIG. 2C to FIG. 2E, the all-point histograms of the current amplitudes before and after perfusing bilirubin revealed that it generated single-channel currents of −2.02±0.05 pA at −40 mV. Further analyses of the dwell-times in open and closed states showed that the opening probability (Po) was significantly increased by bilirubin, which was reversed by FFA. By plotting the amplitude of single-channel currents at different holding potentials, a slope conductance of 76.29±7.85 pS from the linear fit to their bilirubin-driven current-voltage relationship were derived, which is identical to that of its intracellular agonist ADPR (FIG. 2F to FIG. 2G) and consistent with previously reported values (i.e., 60-74 pS). These results strongly support the notion that bilirubin directly gates the TRPM2 channel.

### Example 3

In this embodiment, molecular docking simulation and patch-clamp recordings are used to find the key binding sites for interaction between bilirubin and TRPM2 channels. Specific experimental steps and results are as follows.

1. Molecular docking simulation: Structure of TRPM2 was obtained from Protein Data Bank (PDB ID: 6PUS, 6PUO). 3D structure of bilirubin was obtained from PubChem compound (PubChem CID: 5280352). Molecular docking was generated using the Schrödinger Maestro software suite. Prior to docking, protein was processed using the Protein Preparation Wizard for adding missing residues, removing waters, optimizing H-bond and energy (OPLS3e force field). Ligand was optimized using OPLS3e force field in LigPrep module. Protein and ligand protonation states at pH 7.4±0.2 were sampled using Epik. Ligand was docked to a picked residue in a grid box with dimensions of 25×25×25 Å3. Extra-precision docking (Glide XP) was performed with flexible ligand sampling, and post-docking minimization was performed to generate a maximum of 10 poses per ligand within the Glide program, the docking conformation with a highest docking score was analyzed to identify critical molecular interactions between different ligand moieties and amino acid residuals of the binding pocket for guided site-directed mutagenesis.

2. Electrophysiological experiments (whole-cell patch-clamp recording) is the same as in Example 2

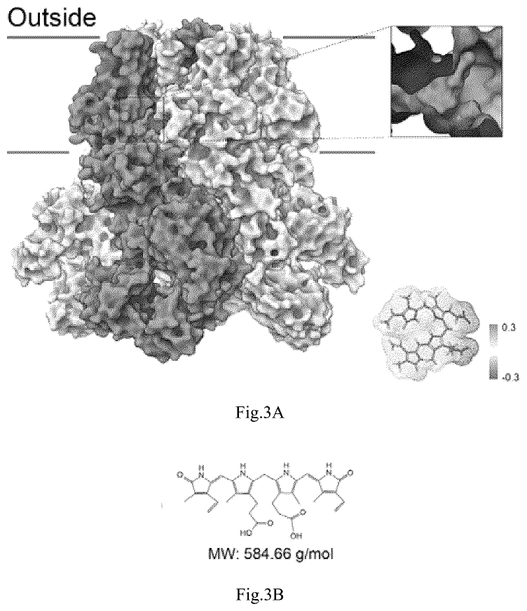

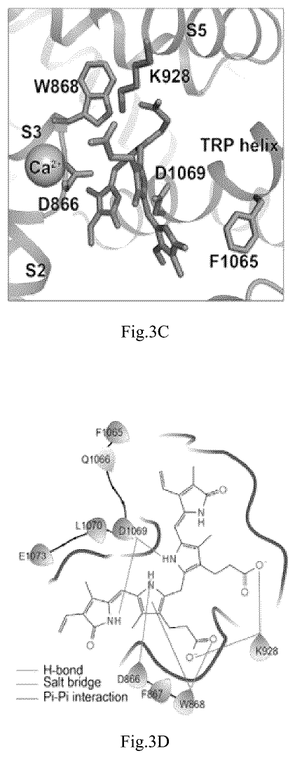

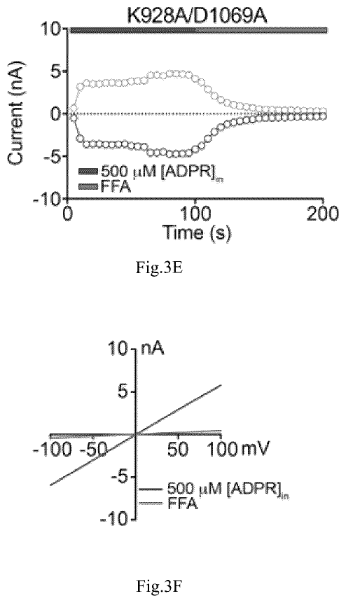

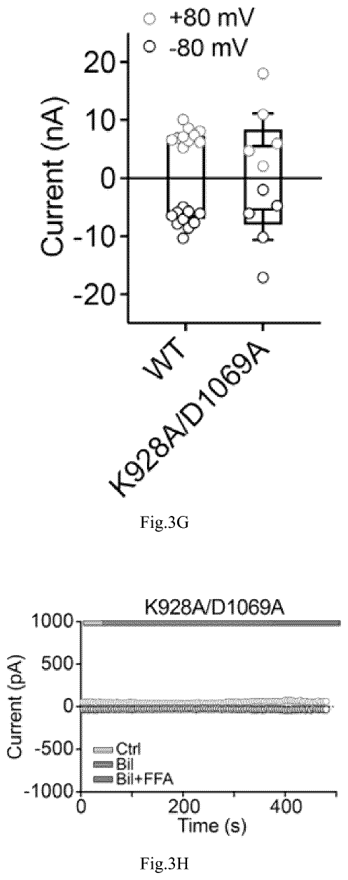

As shown in FIG. 3A to FIG. 3D, there is an optimal cavity near the transmembrane Ca²⁺ binding sites, where bilirubin was surrounded by the S3, S5 and TRP helix. Amino- and carboxyl moieties of bilirubin make four hydrogen bonds (H-bonds) with D866, W868 in S3, and D1069 in the TRP helix and two additional salt bridges with K928 in S5 while its pyrrole moiety forms a strong n-n interaction with W868. The internal space constraints and molecular energetics of chemical bonds between different chemical moieties of bilirubin and the amino acid side chains of the TRPM2 protein were shown in Table 1. According to the analysis of the strength of the interaction, K928 and D1069 represent the strongest binding sites based on bond energy analysis which are most likely to be the key interaction sites. FIG. 3E to FIG. 3I show that intracellular ADPR perfectly retained the agonist capacity to activate this K928A/D1069A double mutant TRPM2 channel with the current amplitude being indistinguishable from that of the WT hTRPM2 channel, which indicates that the double mutations do not perturb the gating and permeation properties of TRPM2 channels via canonical intracellular agonists. In contrast, bilirubin completely lost its ability to activate this double mutant. These results demonstrated that bilirubin activates the TRPM2 channel via a different gating mechanism from canonical intracellular ligands.

TABLE 1
Interaction scores between bilirubin and key residues
of TRPM2 channel in molecular docking

| | E_{Vdw} | E_{coul} | E_{Hbond} | Dis | Eₜₒₜₐₗ |
|---|---|---|---|---|---|
| Residue | (kcal/mol) | (kcal/mol) | (kcal/mol) | (Å) | (kcal/mol) |
| K928 | 1.166 ± 1.113 | 93.804 ± 10.215 | 0 | 1.182 ± 0.154 | 94.032 ± 9.067 |
| D1069 | 7.164 ± 0.543 | 28.723 ± 2.942 | 0.505 ± 0.190 | 2.063 ± 0.160 | 36.392 ± 2.817 |
| D866 | 3.219 ± 1.674 | 32.650 ± 3.531 | 0.246 ± 0.118 | 2.164 ± 0.194 | 36.116 ± 3.687 |
| W868 | 3.984 ± 1.003 | 4.688 ± 2.322 | 0.174 ± 0.188 | 1.989 ± 0.335 | 8.845 ± 1.970 |

### Example 4

In this embodiment, a bilirubin derivative with similar structure (bilirubin disodium salt, BDS) was used to carry out molecular docking simulation, while patch-clamp recordings and cell death assay were used to evaluate antagonistic and neuroprotective effect of A23. Specific experimental steps and results are as follows.

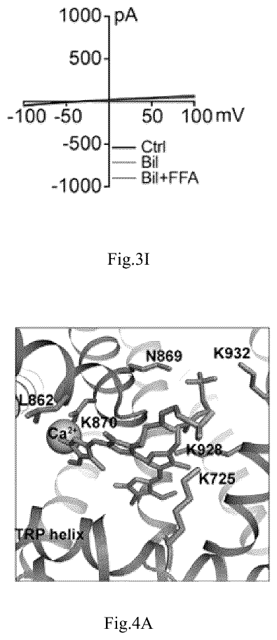

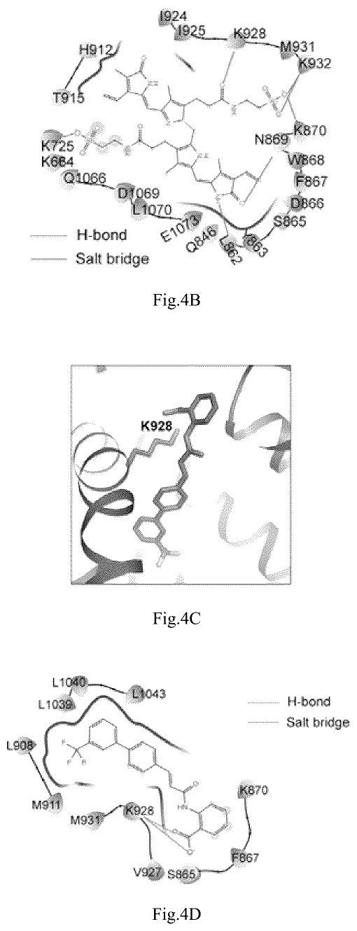

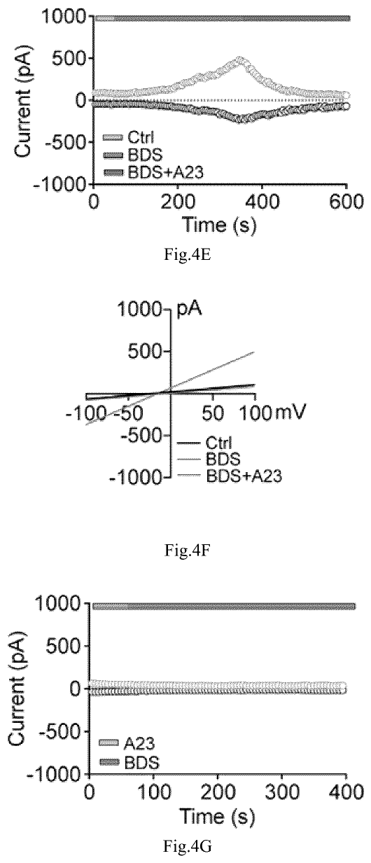

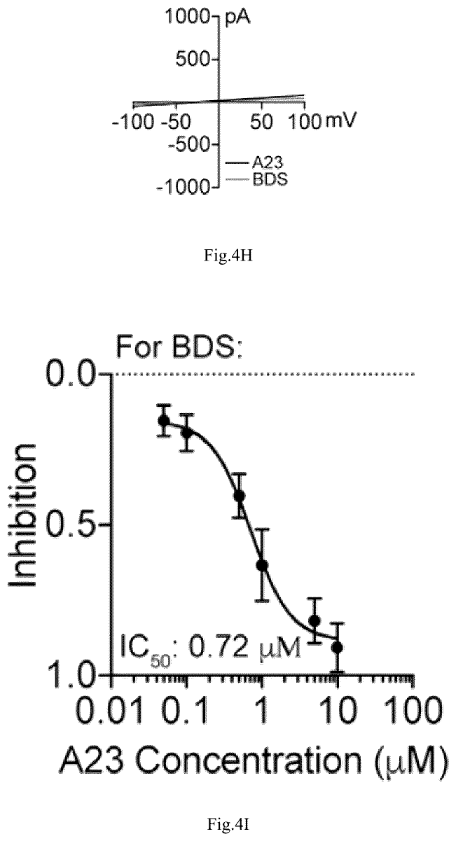

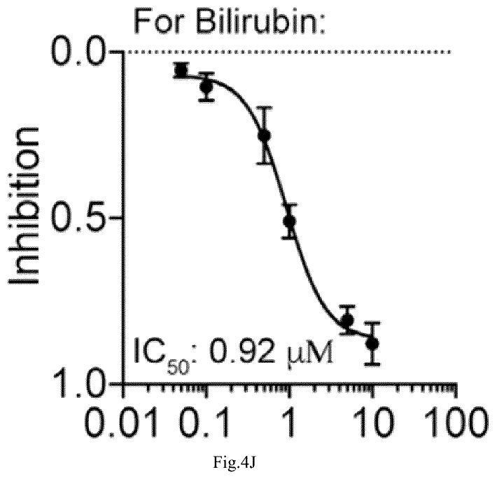

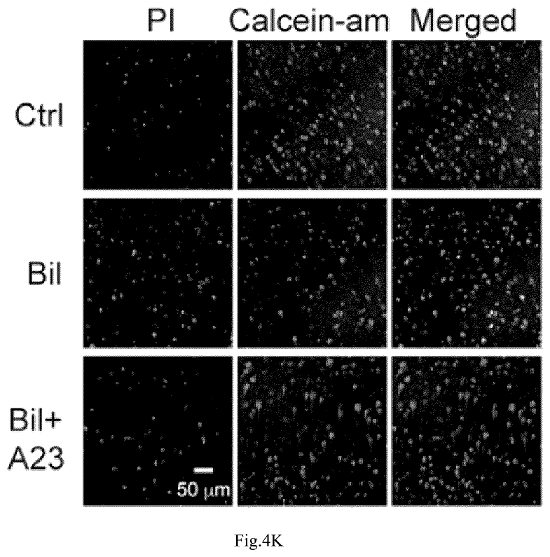

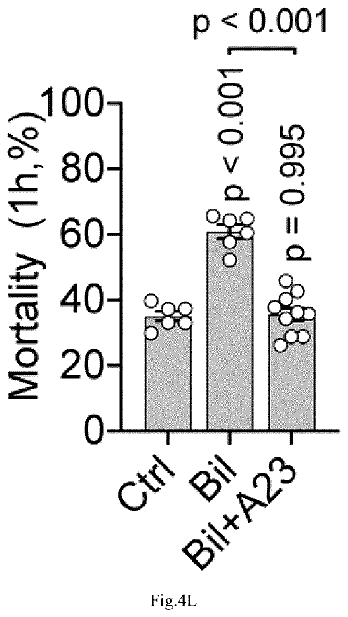

Molecular docking was used to simulate the combination of BDS and A23 (a newly published TRPM2 blocker) with the identified binding pocket for bilirubin, and the specific operation process was the same as that in Example 3. As can be seen from FIG. 4A to FIG. 4D, BDS formed multidimensional interactions with the K725, L862, N869, K870, K928, K932 residues through H-bonds and salt bridges, while A23 formed H-bonds and salt bridges with K928 to optimally fit into the cavity. It exhibited the lowest docking score for the Apo State (6PUO) of TRPM2 (bilirubin −5.04, BDS −4.47, A23 −8.57) and therefore the strongest binding.

Next, to avoid any off-target effects of bilirubin on intracellular gating pathways, we used its water-soluble and membrane-impermeable derivative BDS as an agonist to activate the TRPM2 channel Electrophysiological experiments (whole-cell patch-clamp recording) is the same as in Example 2. As shown in FIG. 4E to FIG. 4H, that A23 completely inhibited the BDS-activated TRPM2 current, A23 not only completely suppressed TRPM2 currents preactivated by BDS, but also prevented its activation when A23 was first applied. Further experiments showed that A23 blocked currents activated by BDS and bilirubin with its IC so being 0.72 μM and 0.92 μM, respectively.

In view of the high affinity between A23 and the bilirubin-binding cavity in the TRPM2 channel, it was next tested whether A23 can reduce the neurotoxicity of bilirubin by in vitro cell death assay. In this assay, surviving and dead cortical neurons were labeled with calcein-am and propidium-iodide (PI), respectively (FIG. 4K), the specific process was as follows:

1. Trpm2^{+/+} mice aged 4-8 weeks were anesthetized by intraperitoneal injection of 1% pentobarbital (55 mg/kg). The brain tissue of the mice was then acutely isolated, and brain slices with a thickness of 150 μm were obtained using a vibratome.

2. The brain slices were incubated with bilirubin (Bil) and bilirubin+A23 (Bil+A23) for 1 hour at 37° C., adding calcein-am (1 μM) and PI (2 μM) for the last 15 minutes, and then washed for three times before being fixed.

3. When the staining was completed, the cells were fixed with 4% paraformaldehyde in a dark container for 40 minutes, and finally confocal imaging was performed. Cell counts were performed using the software of Image-J to calculate the death rate.

The results showed that in adult Trpm2^{+/+} brain slices, bilirubin significantly increased the mortality of cortical neurons after 1 hour of incubation, and A23 fully antagonized this effect of bilirubin (FIG. 4K to FIG. 4L, Ctrl: 35.01±1.49%; Bil: 60.84±2.13%; Bil+A23: 35.27±1.85%). These results indicate that the bilirubin-binding pocket of the TRPM2 channel is an ideal drug target for compounds with A23-like structures to reduce bilirubin-dependent neurotoxicity in cerebral apoplexy and hyperbilirubinemia (jaundice)-related brain injuries.

### Example 5

In this embodiment, D1066A knock-in (KI) mice were used to explore the effect of molecular perturbation of bilirubin-TRPM2 binding cavity on the neurotoxicity of bilirubin. Specific experimental steps were the same as those in Example 1, roughly as follows.

1. Construction of transient middle cerebral artery occlusion (tMCAO) animal model

2. Detection of UCB concentration in serum and cerebrospinal fluid (CSF) of animal model after operation

3. Measurements of cerebral infarct volume in animal models of ischemic stroke

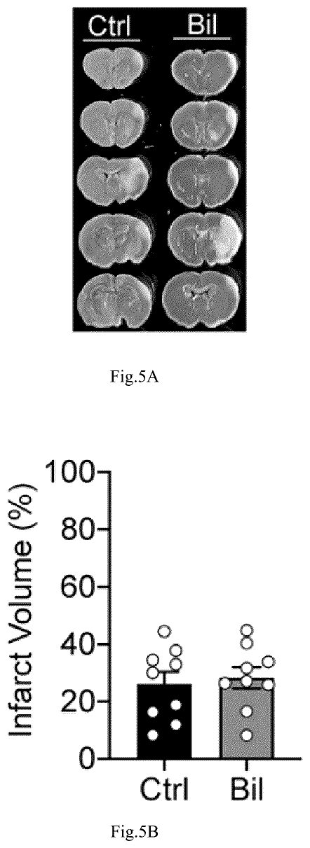

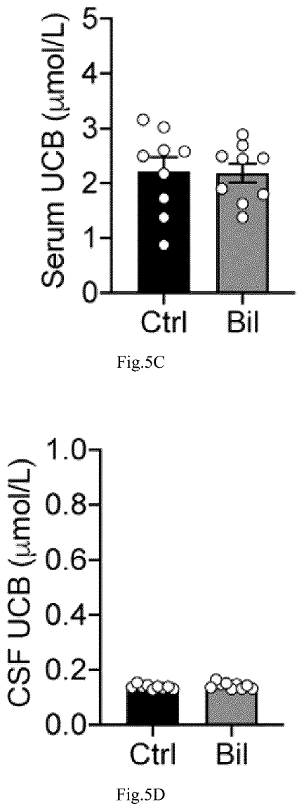

As shown in FIG. 5A to FIG. 5D, the exacerbated ischemic brain injury by bilirubin in tMCAO model was completely ablated in D1066A KI mice, as was the increase in the UCB concentration before and after tMCAO surgery.

In conclusion, these aforementioned results demonstrate that bilirubin and its metabolic derivatives directly binds to and activates the TRPM2 channel via a specific binding cavity in the transmembrane domain and D1069 (the analogous D1066 residue of the mouse TRPM2) is the key amino acid residue for the binding of bilirubin-TRPM2. A23-like compounds targeting this residue can effectively block the activation effect and antagonize neurotoxic damage of bilirubin and its derivatives.

Embodiments of the present invention are described in details for the sake of example only, without limiting the scope of the invention. Those skilled in the art should be able to realize that the many modifications, variations and alternations fall within the scope of the invention. Thus, equivalent alterations and modifications made without departing from the spirit and scope of the present invention shall be considered within the scope of the invention.

What is claimed is:

1. A method for treating cerebral apoplexy and hyperbilirubinemia, the method comprising administering to a subject in need thereof a therapeutically effective amount of the medicine comprising a compound or a reagent for competitively inhibiting or blocking combination of bilirubin and the TRPM2 channel.

2. The method of claim 1, wherein K928 and/or D1069 mutation eliminates channel activating effect of bilirubin and its derivatives.

3. The method of claim 1, wherein the compound or the reagent is competitively combined with the K928 and/or D1069 residue(s) of TRPM2 channel.

4. The method of claim 2, wherein the mutation is a substitution mutation.

5. The method of claim 4, wherein the TRPM2 channel protein has a substitution mutation of the K928 and/or the D1069 residues.

6. The method of claim 5, wherein the TRPM2 channel protein has a substitution mutation of the K928 and D1069 residues.

7. A medicine for treating cerebral apoplexy and hyperbilirubinemia, the medicine comprising a compound or a reagent for competitively inhibiting or blocking combination of bilirubin and the TRPM2 channel, wherein the compound or the reagent acts as a main active component.

8. The medicine of claim 7, wherein the compound or the reagent prevent the bilirubin and its derivatives from activating the TRPM2 channel, the mutation occurs at K928 and/or D1069 residue(s); preferably, the mutation is a substitution mutation; more preferably, the TRPM2 channel has a substitution mutation of the K928 and/or the D1069; more preferably, the TRPM2 channel protein has a substitution mutation of the K928 and the D1069 residues; or the compound or the reagent is competitively combined with the K928 and/or the D1069 residue(s) of the TRPM2 channel and thus prevents the bilirubin from activating the TRPM2 channel.

9. The medicine of claim 7, further comprising a pharmaceutically acceptable carrier or a pharmaceutically acceptable excipient.

10. The medicine of claim 7, routes for administration of the medicine comprise oral, transdermal, intramuscular, subcutaneous or intravenous injections.
